(19) United States
(12) Patent Application Publication
(10) Pub. No.: US 2009/0012354 A1
(43) Pub. Date: Jan. 8, 2009

(54) SYSTEM AND METHOD FOR INTRAVENTRICULAR TREATMENT

(75) Inventor: Malissa J. Wood, Concord, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: The General Hospital Corporation d/b/a Massachusetts General hospital, Boston, MA (US)

(21) Appl. No.: 12/166,609

(22) Filed: Jul. 2, 2008

Related U.S. Application Data
(60) Provisional application No. 60/929,650, filed on Jul. 6, 2007.

(51) Int. Cl. A61F 2/02

(52) U.S. Cl. 600/37

(57) ABSTRACT

Various methods and devices are provided for remodeling a heart's ventricular walls and improving of the function of the atrioventricular valves from within the ventricle or atrium through the use of tensioning structures. In one embodiment, a device for stabilizing a ventricle is provided and can include a superior tension member having a substantially arcuate shape that is sized and configured to improve a functioning of atrioventricular valve leaflets. The device can also include a descending tension member extending inferiorly from at least a portion of the superior tension member that is shaped to correspond to a wall of a ventricular cavity. The device can further include a plurality of anchors provided at least on the descending tension member that have attachment features for holding a wall of a ventricular cavity to the descending tension member.

### RELATED APPLICATIONS

The present application claims priority to U.S. Provisional Application No. 60/929,650 filed on Jul. 6, 2007 and entitled “System and Method for Percutaneous Ventricular Stent and Partial Mitral Annuloplasty,” which is expressly incorporated herein by reference in its entirety.

### BACKGROUND OF THE INVENTION

Congestive heart failure and associated mitral regurgitation is a serious health problem in the United States and around the world. Today, the average American has a 20% chance of developing heart failure in their lifetime. In 2006, nearly 5 million Americans were living with heart failure and an additional 550,000 new cases present each year. Between the years 1979 and 2000, heart failure deaths increased 148% with the direct and indirect costs exceeding $24 billion.

Structural changes observed in patients with cardiomyopathies include dilatation of the left ventricular cavity in response to the increased filling pressures. The dilatation of the ventricle and annulus can lead to functional mitral regurgitation or the presence of mitral regurgitation due to incomplete closure of the valve. The presence of mitral regurgitation can beget further left atrial and left ventricular dilatation. In this setting medical therapies are less likely to be of benefit. The current option available to treat the functional mitral regurgitation is surgical valve repair. While some new results appear promising, the invasive nature of this technique causes many physicians to be reluctant in referring their patients for this procedure.

Functional mitral regurgitation is a marker of adverse left ventricular remodeling, and increased sphericity of the chamber and is associated with worsened outcome. Further, individuals having a myocardial infarction complicated by the appearance of worsened mitral regurgitation have been shown to have increased mortality. The survival in these patients is inversely correlated with the degree of mitral regurgitation.

A myocardial infarction can be initially local in patients with ventricular dysfunction, but can spread throughout the left ventricle as part of the process of deleterious remodeling. Transmural strain may be altered in remote regions of the myocardium, possibly triggered by apoptosis and disruption of the extracellular matrix. One study examined the effect of posterior infarction on overall left ventricular strain and demonstrated that increased transmural shear strain occurs not only in the adjacent myocardium but also at sites remote from the localized infarction.

Currently available treatments for chronic ventricular dysfunction depend upon the etiology of the dysfunction and include coronary artery bypass grafting, mitral and tricuspid repair, and ventricular reduction procedures. The treatment of functional atrioventricular valvular regurgitation and associated ventricular dysfunction include placement of annuloplasty rings. Treatment of ischemic mitral regurgitation and the associated left ventricular dysfunction include the placing of undersized mitral annuloplasty rings. Recently, it has been suggested that the placement of uniquely shaped mitral annuloplasty rings may lead to reduction in degree of mitral regurgitation and improvement in left ventricular systolic function. The most common approach to the treatment of functional mitral regurgitation at the present time is mitral annuloplasty with or without coronary artery bypass grafting. In some patients ventricular remodeling surgery is also performed (Dor procedure/modified Batista procedure). Despite the relatively low mortality in this group of patients, the long-term results have not been satisfactory. One retrospective analysis considered the possible benefit of mitral annuloplasty in individuals with left ventricular ejection fraction (LVEF)<30% and 3-4+ mitral regurgitation. This study found no survival benefit with annuloplasty, both with and without coronary artery disease. One possible reason for the lack of improvement in these patients was the heterogenous nature of the patient population with the inclusion of patients with both ischemic and non-ischemic cardiomyopathies.

The process of post-myocardial infarction ventricular remodeling starts immediately after myocardial infarction and is then associated with further expansion of the infarcted territory. The extent of expansion is largely dependent upon the degree of myocardial necrosis that occurs in the setting of coronary ischemia which is related to duration of ischemia. Even in individuals treated with primary stenting there may be myocardial necrosis if the stenting is not performed within 60 minutes of onset of ischemia. The morbidity and mortality associated with post-myocardial infarction is substantial.

Accordingly, techniques and devices are provided for addressing dilatation of the atrial and ventricular cavities, as well as functional mitral regurgitation in patients with cardiomyopathies.

### SUMMARY OF THE INVENTION

The invention described herein includes a number of devices and methods that relate to the remodeling of a heart's ventricular walls and improvement of the function of the atrioventricular valves from within the ventricle or atrium through the use of tensioning structures. In a first aspect, a device for stabilizing a ventricle is provided and includes a superior tension member having a substantially arcuate shape that can be sized and configured to improve a functioning of atrioventricular valve leaflets. The device also includes a descending tension member extending inferiorly from at least a portion of the superior tension member that is shaped to correspond to a wall of a ventricular cavity. The device further includes a plurality of anchors provided at least on the descending tension member that can have attachment features for holding a wall of a ventricular cavity to the descending tension member.

In another aspect, a method is provided for improving the function of a damaged or diseased heart and includes inserting a device into the ventricle of a patient's heart. The device can include a superior arcuate member, a descending element extending inferiorly from the superior arcuate member, and a plurality of anchors provided at least on the descending element. The method can further include locating the superior arcuate member below an atrioventricular valve and attaching the anchors to a wall of the ventricle, thereby causing the wall to substantially conform to the descending element.

In a final aspect, a method is provided for remodeling a portion of a patient's heart and includes delivering a tension patch through a vein or artery into a ventricle, deploying the tension patch within the ventricle, and anchoring the tension patch on an extended ventricular wall so that the wall is drawn inward.

Specific embodiments of any of these aspects can include a device wherein the superior tension member is a half ring. In other embodiments, the descending tension member can be a mesh formed of intersecting wires, and the anchors can be attached to the mesh at points of intersection between the wires. In one embodiment, the descending tension member can be a mesh formed substantially in the shape of at least a partial cone. The anchors can be provided only on a portion of the mesh that is configured to contact a wall of a ventricular cavity. In other embodiments, a portion of the mesh can form the superior tension member.

While the anchors can have many configurations known in the art, in one embodiment each of the plurality of anchors can form an elongate member having a pointed end opposite to an end attached to the mesh. Each anchor can be positioned in a plane parallel with a plane of the mesh in a delivery configuration. Each of the plurality of anchors can also extend from the mesh at an angle greater than about 45 degrees with respect to a plane of the mesh in a piercing configuration. Further, in some embodiments, each of the plurality of anchors can include at least two attachment elements extending in different directions from the elongate member at an angle greater than about 90 degrees and less than about 180 degrees to grab tissue in a first securing configuration. The two attachment elements can extend in different directions from the elongate member at an angle less than about 90 degrees and greater than about 0 degrees to secure the tissue to the device in a second securing configuration. In other embodiments, the plurality of anchors can have a hooked shape when in a securing configuration within tissue.

The device can be expandable to any shape known in the art, and in one embodiment the device can be expandable into a deployed shape substantially in the form of a quadrilateral from an introduction shape having a smaller cross-sectional dimension than the deployed shape. In another embodiment, the device can be expandable into a deployed shape substantially in the form of a half cylindrical shape from an introduction shape having a smaller cross-sectional dimension than the deployed shape. In still another embodiment, the device can be expandable into a deployed shape substantially in the form of a triangle from an introduction shape having a smaller cross-sectional dimension than the deployed shape.

The device can be formed of any biocompatible material known in the art, including but not limited to nitinol, deformable stainless steel, spring stainless steel, a polymer, and/or a bioresorbable material. The device can also be formed of or coated with a therapeutic agent such that the therapeutic agent can be delivered directly into the myocardium.

The device can be located anywhere within the heart and in one embodiment the superior tension member can be positioned inferiorly to the mitral valve and the descending tension member can be shaped to correspond to a posterior wall of the left ventricle. In some embodiments, the device can be inserted in a first, introduction state, and methods can further include the step of expanding the device to a second, deployed state within the ventricle.

In one embodiment, the device can include an expandable mesh and the device can be inserted over a balloon catheter and over a guidewire anchored in an atrial appendage. The method can further include the step of expanding the device by inflating the balloon. The device can be inserted into the heart in any number of ways known in the art. For example, the device can be inserted into the ventricle using a retrograde transaortic approach, a transseptal anterograde approach, a coronary sinus anterograde approach, a transvenous approach, and/or transarterial approach.

While the anchors can be deployed and secured in any number of ways, in one embodiment, attaching the anchors can include a step of expanding the anchors from an introduction shape to a deployed shape for piercing tissue. Attaching the anchors can also include a step of piercing a wall of the ventricle and deploying attachment members from the anchors within the wall to form an open configuration. Attaching the anchors can further include a step of moving the attachment members from the open configuration to a closed configuration to pull the tissue against the device.

The exemplary devices and methods presented herein are particularly advantageous for delivering a therapeutic agent directly into the myocardium. In one embodiment, the method can include coating the device with or forming the device of a fibrous matrix and the therapeutic agent can be one of an endothelial growth factor, a gene therapy, or a vasoactive substance. The method can also include closing a defect within the ventricular myocardium using a fibrous matrix formed on the device. In one embodiment, the step of expanding the device can include expanding a device formed of a shape-memory material. In other embodiments, the superior arcuate member can be located below the mitral valve and the anchors can be attached to a posterior wall of the left ventricle. In methods in which a tension patch is delivered, the tension patch can be delivered using a balloon catheter.

### BRIEF DESCRIPTION OF THE DRAWINGS

The invention will be more fully understood from the following detailed description taken in conjunction with the accompanying drawings, in which:

FIG. 1A is a cross-sectional view of a healthy human heart;

FIG. 1B is a cross-sectional view of a healthy left ventricle and atrium;

FIG. 1C is a cross-sectional view of a diseased left ventricle and atrium;

FIG. 1D is a diagram of an inverted left ventricle and mitral valve illustrating a normally functioning left ventricle and mitral valve;

FIG. 1E is an diagram of an inverted left ventricle and mitral valve illustrating an abnormally functioning left ventricle and mitral valve;

FIG. 2 is a front view of one embodiment of a tension member having anchors formed thereon;

FIG. 3A is a side view of one embodiment of the anchor of FIG. 2, the anchor having a straight piercing portion;

FIG. 3B is a side view of another embodiment of the anchor of FIG. 2, the anchor having an open configuration;

FIG. 3C is a side view of still another embodiment of the anchor of FIG. 2, the anchor having a hook shape;

FIG. 4A is a perspective view of one embodiment of a tension member having a superior arcuate element and a descending element with anchors formed thereon;

FIG. 4B is a perspective view of another embodiment of a tension member having a superior arcuate element and a descending element with anchors formed thereon;

FIG. 5 is a front view of a balloon catheter having one embodiment of a tension member positioned thereon for insertion into the heart;

FIG. 6A is a cross-sectional view of a left ventricle having a balloon catheter and one embodiment of a tension member inserted using a retrograde transaortic approach.

FIG. 6B is a cross-sectional view of the left ventricle of FIG. 6A illustrating expansion and deployment of the tension member within the ventricle;

FIG. 6C is a cross-sectional view of the left ventricle of FIG. 6A illustrating securement of the tension member to the posterior wall thereby reshaping the wall and annulus;

FIG. 7A is a side perspective view of one embodiment of an expanded tension member having anchors deployed for insertion into tissue;

FIG. 7B is a side perspective view of the embodiment of FIG. 7A illustrating the anchors piercing the posterior ventricular wall;

FIG. 7C is a side perspective view of the embodiment of FIG. 7A illustrating the attachment members being deployed within the posterior wall in an open configuration within the tissue;

FIG. 7D is a side perspective view of the embodiment of FIG. 7A illustrating the attachment members being activated to a closed configuration within the posterior wall to draw the wall to the tension member;

FIG. 8A is a diagram of an inverted diseased left ventricle and mitral valve as an exemplary tension member is positioned on a posterior wall; and

FIG. 8B is a diagram of an inverted healthy left ventricle and mitral valve as the tension member of FIG. 8A has remodeled the wall and the mitral valve.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices that relate to the remodeling of a heart's ventricular walls and improvement of the function of the atrioventricular (AV) valves from within the ventricle or atrium through the use of tensioning structures. Methods and devices are also provided relating to the use of tensioning structures for the direct myocardial delivery of therapeutic agents. The methods and devices disclosed are particularly advantageous as they allow for devices that can be introduced into the heart percutaneously and that can provide support and remodeling directly to the heart from within a particular chamber.

In one embodiment, methods and devices are provided that relate to reducing ventricular dimensions in patients with dilated cardiomyopathies. A tension device can be used to reduce the intraventricular dimension in diastole, thereby reducing the wall stress on the ventricle and allowing remodeling of the chamber. In particular, a tension device can limit myocardial stretch by locally reinforcing the ventricle tissue and limiting stress placed on the tissue by diastolic filling pressures. Direct remodeling of the chamber coupled with a reduction in AV valvular regurgitation can provide significant improvement in ventricular function.

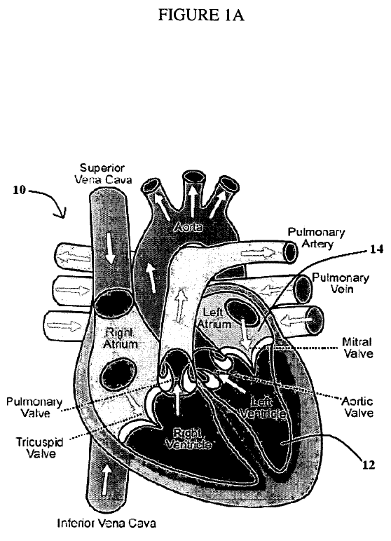

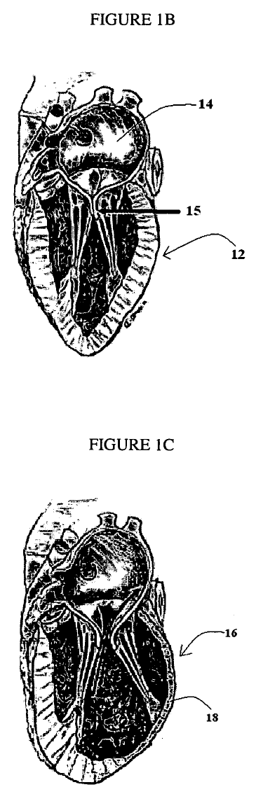

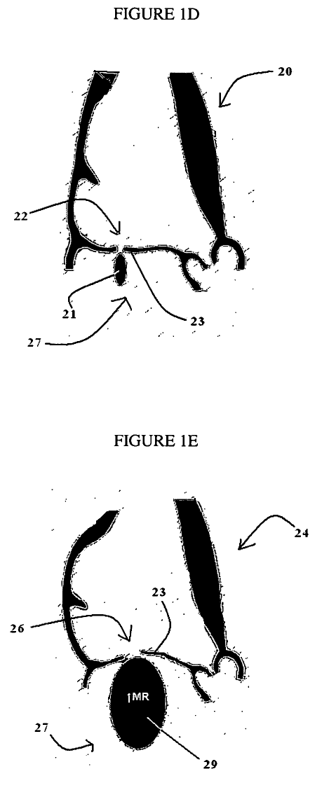

With initial reference to FIGS. 1A and 1B, an anterior cross-sectional view of a healthy heart 10 and a healthy left ventricle 12 and atrium 14, respectively, is shown. The heart and left ventricle shape is associated with a heart that is functioning properly without undue stress on the walls or valves. FIG. 1C is a cross-sectional view of a diseased (enlarged) left ventricle 16. A portion of the left ventricle 18 is shown to stretch as the left ventricle wall is stretched, and ultimately remodeled, due to increased diastolic filling pressures exerted on the diseased tissue following, for example, a myocardial infarction. A radial and axial expansion that is experienced within the heart leads to stretching or degenerative remodeling and concomitant organ enlargement. This enlargement can be localized along the anterior or posterior wall of the left ventricle, can be located or extend septally, can include the right ventricle, and/or can involve the mitral valve annulus 15.

While certain of the figures and associated description specifically address dilation in the left ventricle, a person of ordinary skill will recognize that similar changes can occur in the right ventricle—with or without associated left ventricular dysfunction. Accordingly, in distinct aspects of the invention, the devices and techniques described can be applied in the left ventricle, the right ventricle, or both ventricles. For that reason, the present application includes the diagrammatic cross-section of the human heart of FIG. 1A including right and left sides and uses anatomical terms consistent with this figure for describing the application of devices and methods of the invention in the right ventricle as well as in the left.

In the case in which ventricular expansion occurs due to either pressure or volume overload of the myocardial tissue, the AV valve annulus can also stretch and cause functional valvular regurgitation. FIG. 1D illustrates an inverted view of a healthy left ventricle 20 and a normally functioning mitral valve 22 in which the mitral valve leaflets 23 are able to close sufficiently and prevent blood 21 from flowing back into the left atrium 27. FIG. 1E is an inverted view of a distended left ventricle 24 and an abnormally functioning mitral valve 26 in which blood 29 is leaked from the left ventricle 24 back into the left atrium 27 due to the mitral valve leaflets 23 being unable to close and seal properly. Direct remodeling of heart chambers coupled with a correction of valve functioning can provide significant improvement in atrial and ventricular function.

In general, all tensioning structure embodiments of the present invention can include one or more tension patches or tension members and one or more anchor members for securing a tension member to a chamber wall or tissue surrounding a valve. These components are designed to be able to work in concert in order to facilitate and provide palliative or therapeutic cardiac reinforcement in the cardiac valve annulus, myocardium, and valve leaflets, although any other area within the heart, including, but not limited to, intravascular conduits and chordae tendinae, can benefit from the methods and devices described herein. A number of embodiments of the present invention are provided mainly in the context of tensioning structures positioned and anchored within chambers to provide cardiac muscle support and reinforcement. The primary targets for the tensioning structure embodiments described herein, can include, but are not limited to, in or near the left and right ventricles and/or in or near the left and right atrium, as well as in or near the mitral valve and/or the tricuspid valve. A person skilled in the art will appreciate the various other locations within the heart than can benefit from the use of the tensioning structure embodiments described herein.

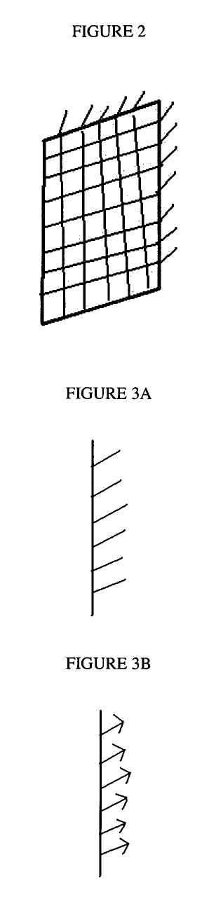

Referring now to FIG. 2, a tension patch or tension member 30 is provided that can generally be formed of a series of interconnecting components 32 that can be formed from wires or fibers into a matrix or mesh 36. As shown, one or more securing members, attachment means, or anchors 34 can be attached to the mesh 36 as will be described in more detail below. The tension member 30 can generally be inserted percutaneously into a chamber of the heart, for example, the left ventricle, and can be anchored directly into or through myocardial tissue to provide reinforcement of the left ventricle wall.

The tension member 30, and thereby the interconnecting components 32, can have any amount of flexibility or rigidity as needed for a reduced dimension delivery into the heart and an expansion and conformation to a ventricular or atrial wall, or other location, once in position within the heart. The interconnecting components 32 can form any sized mesh 36 as needed, and apertures 38 forming the mesh 36 can be large or small with respect to the tension member 30. Further, the wire or fiber that forms the interconnecting components 32 can have any diameter or rectangular cross-sectional dimension depending on the flexibility, rigidity, and/or strength required of the tension member 30. For example, if a very flexible tension member 30 is required, or if only a therapeutic delivery device is needed, the interconnecting components 32 can be smaller in diameter or dimension. If a rigid tension member 30 is required to remodel a particularly distended section of the heart, for example, the interconnecting components 32 can have a larger dimension to provide the needed strength and/or rigidity. A person skilled in the art will appreciate that any dimensional considerations are interdependent with the specific materials used in forming the tension member 30. For example, a particular material may provide a tension member 30 that is both flexible and rigid as needed without requiring a dimensional alteration to the interconnecting components 32.

The tension member 30 can have any size and shape as needed for a particular application. For example, the tension member 30 can be generally flat with any shape known in the art such as a rectangle, circle, triangle, quadrilateral, etc. In other embodiments, the tension member 30 can have a curvature associated with a specific shape to conform to a portion of a ventricle wall, atrial wall, or valve. The tension member 30 can have the shape of at least a partial cone, at least a partial cylinder, at least a partial sphere, etc., as well as any combination of curvatures and shapes as needed to conform to a particular area of the heart.

In some embodiments, the tension member 30 can have a size and shape such that it can be used as a patch or band aid to remodel a specific and/or small area of a ventricular wall. In other embodiments, the tension member 30 can have a size and shape corresponding to an entire chamber within the heart such that it can remodel the chamber. The tension member 30 can also be connected with additional tension members that may have a different size and shape than the tension member 30. For example, the tension member 30 can be connected with an arcuate tension member, as will be described below, that can have a size and shape adapted to reshape and provide support to a particular valve within the heart. As the arcuate member reshapes and provides support for a valve, the tension member 30 can reshape a ventricle or atrial wall adjacent to the valve.

As noted above, in some embodiments, the tension member 30 can have at least one securing member, attachment means, or anchor 34. In general, the anchors 34 can secure the tissue to the tension member 20 to allow reshaping of the tissue. In other embodiments, the anchors 34 can aid with cinching/compression of the local tissue region to reduce wall stress while mitigating over-expansion of the tissue. Also, the anchors 34 can import or help to exert an elastic recoil effect during wall motion of the heart. That is, the tension member 30 can be fixed within a particular chamber by frictional forces imposed upon the wall by the anchors 34 to maintain position of the structure in spite of cardiac wall motion.

As shown in FIG. 2, in one embodiment, the tension member 30 can have a plurality of anchoring members 34 extending therefrom that can be configured for engaging, piercing, grasping, and/or securing tissue as needed. The anchors 34 can provide securement of the tension member 30 to the tissue so that the tension member 30 can reshape and provide support to a particular area within the heart. In some embodiments, the anchors 34 can be attached at the intersection of the interconnecting components 32 and extend therefrom. In other embodiments, the anchors 34 can be attached to a center portion of the straight sections of the interconnecting components 32.

In some embodiments, the anchors 34 can be formed integrally with the tension member 30. In other embodiments, the anchors 34 can be formed as separate components. For an integrated configuration, anchors 34 can be fabricated from one or more strands of material that can form any anchor geometry as needed and simply be an extension of one or more strands that produce the tension member 30. For a nonintegrated condition, any anchor configuration can be bonded or attached to the tension member 30. For example, the anchors 34 can be formed from a mesh or braid of raw material strands that are attached or tied to the tension member 30 at the intersection of the interconnecting components 32. Alternatively, the anchors 34 can be glued, ultrasonically welded, spot welded, soldered, or bonded with other means, depending on the types of materials used, to the tension member 30. In other embodiments, the anchors 34 can be fabricated from a tube or other raw material geometry laser cut into the desired shape and attached or bonded to the tension member 30. It should be noted that laser cutting, chemical etching, water-jet cutting, or other cutting mechanism can be used to create the anchors 34 and the tension member 30 as an integrated unit from a single piece of raw material (tube stock, sheet stock, or other geometry). A person skilled in the art will appreciate the various ways that the anchors 34 can be attached to and/or formed with the tension member 30. For example, exemplary methods of forming and attaching anchoring components to tension members can be found in U.S. Pat. No. 7,144,363 entitled, “Systems for Heart Treatment,” which is incorporated herein by reference in its entirety.

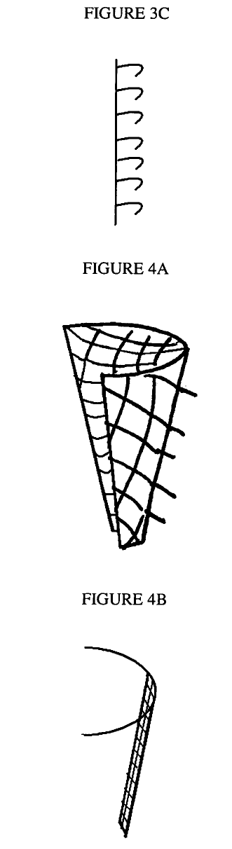

Referring now to FIGS. 3A-3C, the anchors 34 can have any shape or form that is able to pierce, engage, and/or grab tissue and hold it. In one embodiment shown in FIG. 3A, the anchors 34 can be in the form of substantially rigid elongate portions 42 that have a piercing tip 44 on an end opposite to an end 46 attached to the tension member 30. In an embodiment shown in FIG. 3B, the anchors 34 can have two attachment members or features 48a, 48b extending in different directions from an elongate portion 50. In one embodiment, once the anchors 34 are positioned within the tissue, the anchors 34 can take a first securing configuration in which attachment features 48a, 48b, associated with the anchors 34, can be deployed. The features 48a, 48b can be deployed such that each feature 48a, 48b extends in different directions from an elongate portion 50 in an open configuration at an angle that is greater than about 90 degrees and less than about 180 degrees. The anchors 34 can also have a second securing configuration or closed configuration in which the features 48a, 48b can snap or move from the first securing configuration to each extend from the elongate portion 50 at an angle that is less than about 90 degrees and greater than about 0 degrees to form an umbrella shape or spear shape as shown in FIG. 3B. This snapping or moving from the first securing configuration to the second securing configuration creates a retrograde action on the tissue such that the tissue is pulled or drawn inward against the tension member 30 and secured thereto. In an embodiment shown in FIG. 3C, the anchors 34 can have a hook-shaped portion 52 such that the hook 52 resists pullout once inserted into tissue. In other embodiments, the anchors 34 can have a sine-wave shape or other curved shape to create a frictional engagement with the tissue. Any variations and combinations of anchors 34 can be used, including variations on the straight anchor, umbrella anchor, and hook shaped anchor. In all embodiments described herein, the anchors 34 can be configured to be removable from the tissue such that the tension member 30 can be removed and repositioned or withdrawn completely.

In all anchor embodiments, the anchors 34 can have multiple configurations based on whether the tension member 30 is being delivered to an area of the heart, deployed within the heart, or secured to tissue. Any mechanism known in the art can be used to allow multiple configurations for the anchors 34. A mechanical mechanism can be used to deploy features of the anchors 34 once the tension member 30 is inside the heart and/or once the anchors 34 are inserted in tissue. Shape memory and heat activated materials can be used to deploy features of the anchors 34 for different configurations. For example, after insertion of the tension member 30 into a particular chamber, a balloon can be used to expand the tension member 30 and thereby the anchors into a plastically-deformed or shape-memory configuration. In addition, using the balloon expansion method, the anchors 34 can be sequentially deployed using the same or multiple balloons. Alternatively, self-expanding anchors 34 can be released from an external, compressive sheath that maintains the anchors 34 in a compressed, low profile state during positioning predeployment.

In one embodiment, such as the embodiment shown in FIG. 3B, the two portions 48a, 48b can be positioned parallel with the elongate portion 50 during an insertion or delivery configuration of the tension member 30. All three portions 48a, 48b, 50 can be folded down into a low profile position parallel with a plane of the tension member 30 such that the entire device can be placed within a sheath or other mechanism for delivery through a catheter, as will be described below. Once inside the heart, the tension member 30 can be expanded, for example by a balloon catheter, and the three portions can extend outward from the tension member 30 at an angle generally greater than, for example, about 45 degrees with respect to the tension member 30. The anchors 34 can be inserted into tissue in this configuration as described above.

In other embodiments, such as the embodiments shown in FIGS. 3B and 3C, the anchors can again be inserted into the heart with a low profile configuration generally parallel to the plane of the tension member 30. Once the tension member 30 is expanded inside the heart, the anchors 34 can deploy out into a tissue piercing configuration. Once inside the tissue, heat from the tissue or elsewhere can cause deformation of anchors 34, which can be formed of a shape-memory material, to a pre-determined shape, such as an umbrella or a hook, thereby preventing pullout. Heat-activated shape memory can be used in any anchoring configuration to allow delivery into the heart and piercing of tissue, followed by securing or anchoring within the tissue. A person skilled in the art will appreciate the various ways in which the anchors can be configured to retain or change their shape once inside the heart.

Referring now to FIGS. 4A and 4B, an exemplary tensioning structure 60 is shown that can be used in modifying the shape of the ventricular wall immediately inferior to the atrioventricular valve. In one embodiment, a tension patch or tension member 62 is provided for stabilizing a left ventricle and mitral valve within a patient's heart. The tension member 62 can include a superior tension element 64 with a substantially arcuate shape and a descending tension element 66 having a substantially half, frusto-conical shape, cylindrical shape, triangular shape, or a curved quadrilateral shape. The descending element 66 can extend inferiorly from at least a portion of the superior element 64 and can be shaped to correspond to a posterior wall of a left ventricular cavity. The tension member 62 can also include a plurality of anchors 34 provided at least on the descending element 66 that can have attachment features for engaging and holding tissue within the left ventricle. More particularly, once inserted within the left ventricle of the heart, the anchors 34 extending from the descending member 66 can engage a posterior wall of the left ventricular cavity and hold the posterior wall to the descending element 66 to thereby reshape the wall. In another embodiment, the superior element 64 can also have anchors 34 extending therefrom that can engage the myocardium inferior to the AV annulus and can thereby assist in anchoring the device.

The superior element 64 can have a generally arcuate or semi-circular shape, although any portion of a circle or portion of an oval shape can optionally be used. In some embodiments, a full circular or oval ring can be used. In the embodiment shown in FIG. 4A, the descending element 66 extends inferiorly from an entire length of the superior element 64 to form a half frusto-conical shape, a curved quadrilateral shape, or a generally half inverted cone shape that terminates in a plane parallel to the base of the cone. In some embodiments, for example the embodiment shown in FIG. 4B, a descending element 70 can extend from only a portion of a superior element 72 to form a portion having a width that is smaller than a width of the superior element 72. In other embodiments, the superior element and descending element can be a single combined element with the superior element simply being a thickened portion of mesh compared to the descending element. In other embodiments, the superior element can be a separate half-ring like component that can be permanently attached to or removably attachable to the descending member. In any embodiments, the superior arcuate element can generally provide support for and remodeling to a valve, such as a mitral valve, by engaging tissue around the valve and providing tensional support thereto.

Any of the anchoring members 34 described herein can be attached to a descending element and/or a superior element to allow attachment to the left ventricle wall and the infraannular myocardium. In general, a series of anchors 34 can be placed at inferior, mid, and superior portions of the tension member 30. The anchors 34 can be placed in line with one another along a particular spine of the tension member 30 or can be offset or placed randomly as needed. The number of anchors 34 disposed on the tension member 30 can depend on a specific situation, but can be sufficient to provide enough tensile strength or support to remodel the tissue as needed. For example, 1, 2, 3, 4, 5, 6, 7, 8 or more anchors 34 can be used along a descending element to attach it to the ventricle wall, although the number of anchors 34 will preferably between 3 and 6. Similarly, 1, 2, 3, 4, 5, 6, 7, 8 or more anchors can be provided on a superior arcuate element to secure the superior element to tissue around, for example, the AV valve. In general, anchors 34 can be positioned on a descending element and a superior element at any point in which tension will be applied to the ventricular wall or AV valve annulus. A person skilled in the art will appreciate that any number and type of anchors 34 can be positioned anywhere on the tension member 30 as needed.

In general, the tension members, anchors, and any additional components described herein can be formed from any material known in the art that is biocompatible. In one embodiment, the tension member 30 and the anchors 34 are preferably fabricated from biocompatible materials commonly used in medical implants, including nickel-titanium or nitinol (especially, for example, for self-expanding or thermally-actuated tension members and anchors), deformable stainless steel, spring stainless steel, and/or other metals and alloys capable of being deformed using balloon catheters or other expansive means, such as self-expansion. Alternatively, or in addition, the tension member 30 and anchors 34 can be fabricated from superelastic polymers, flexible or deformable polymers such as urethane, expanded PTFE, or stiff materials such as FEP, polycarbonate, etc. A person of skilled in the art will appreciate that the tension member 30 and anchors 34 can be formed of the same material or a mixture of materials and that any suitable material known in the art can be used.

In other embodiments, the tension member 30 and the anchors 34 can be formed of a bioresorbable material. A variety of resorbable, biocompatible materials can be used and in one embodiment, polymers may be employed for manufacturing the tension member 30 and the anchors 34. Homopolymers and copolymers such as those disclosed in U.S. Pat. No. 5,412,068 entitled “Medical Devices Fabricated from Homopolymers and Copolymers Having Recurring Carbonate Units,” incorporated herein by reference in its entirety, are appropriate for resorbable tension members. Other polymers can include, but are not limited to, dextran, hydroxyethyl starch, gelatin, derivatives of gelatin, polyvinylpyrolidone, polyvinyl alcohol, poly[N-(2-hydroxypropyl)methacrylamide], polyglycols, polyesters, poly (orthoesters), poly (ester-amides) and polyanhydrides. In one embodiment, the tension member 30 and the anchors 34 can be fashioned from polyesters such as poly (hydroxy acids) and copolymers thereof, poly (.epsilon.-caprolactone), poly (dimethyl glycolic acid), or poly (hydroxy butyrate). In another embodiment, the tension member 30 and the anchors 34 can be manufactured of polymers of D,L-polylactic acid, L-polylactic acid, or glycolic acid, or copolymers (two or more) of D,L-polylactic acid, L-polylactic acid, and glycolic acid. Such polymers may be manufactured and configured as disclosed, for example, in U.S. Pat. No. 5,133,755 entitled “Method and Apparatus for Biodegradable, Osteogenic, Bone Graft Substitute Device,” incorporated by reference herein in its entirety. A person skilled in the art will appreciate the variation of materials that can be chosen and will further appreciate that materials can be chosen based upon the amount of time the tension member should provide remodeling and support before complete degradation occurs.

In general, the performance of the tensioning structure depends upon and can be tailored to the desired features. For example, when column strength is required, superelastic materials or other alloys or metals can be used to form the tension member and anchors. When pure tension is required and the tensioning structure is to be deployed through tortuous access points, more flexible materials such as expanded PTFE, polyester, or other suture type materials can be used. When absorption or biological integration is desired over a period of time, biological materials such as strips of pericardium or collagen, or absorbable materials can be used.

In one embodiment, the tension member 30 can be formed of a shape-memory material, for example, nitinol, titanium or stainless steel, or a biodegradable polymer and can be wrapped tightly around a deflated, expandable device. For example, the tension member 30 can be wrapped around an expandable high pressure balloon 80 as used with a balloon catheter 82, as is typically known in the art. It will appreciated, however, that any delivery system catheter can be used. The balloon 80 can be made of any biocompatible material known in the art, preferably elastomeric, including but not limited to silicone rubber, natural rubber, polyvinyl chlorides, polyurethane, copolyester polymers, thermoplastic rubbers, silicone-polycarbonate copolymers, polyethylene ethyl-vinyl-acetate copolymers, woven polyester fibers, or combinations of these. The balloon catheter 82 can be generally configured to remain in a deflated state while inserted through the body and can be configured to be expandable to produce an outward pressure against the tension member 30 to cause it to expand once in position within the ventricular chamber.

In general, deployment and delivery of any tensioning structure to a required location within the heart can be achieved using any methods known in the art, but can generally be achieved using a catheter-based approach to access the endocardium, vasculature, myocardium, or epicardium. Described below are methods that relate to deploying tension members into chambers within the heart, including the left and right ventricles, to reinforce the chamber walls about infarcted/ischemic regions, as well as to reinforce the mitral valve annulus to address mitral regurgitation or other insufficiencies.

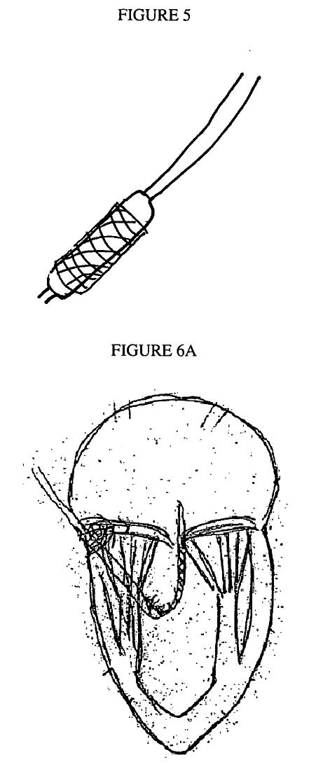

Referring now to FIG. 5, an exemplary insertion method will be described. In general, a tension member 30 can have a delivery or insertion configuration in which the tension member 30 is configured for safe delivery through the body and into the heart in a compacted condition. A guidewire 84 can be used in conjunction with the balloon catheter 82 to facilitate insertion into the heart, as is described below.

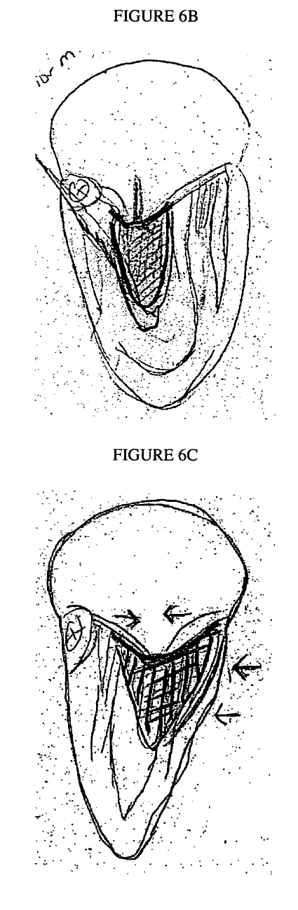

In particular, in one embodiment as shown in FIG. 6A, a sheath 86 such as, for example, an 8 French sheath, can be inserted through the aorta 88 to facilitate delivery of the tension member 30. The guidewire 84 can be inserted through the aorta 88 and aortic valve 90 and into the left ventricle 92. The guidewire 84 can be guided up through the mitral valve 94 and into the left atrium 96 and can be secured thereto by any securing mechanism known in the art. Once the guidewire 84 is in place, the balloon catheter 82 can follow the guidewire 84 through the sheath 86 and into the left ventricle 92 until a top or leading portion of the balloon catheter 82 is in a position towards the posterior wall 98 of the ventricle 92 between the papillary muscles 100 and just below the mitral valve 94. The balloon 80 can be expanded such that pressure is exerted on the tension member 30 to cause it to expand outward and take a predetermined shape, for example, through the use of a shape memory material. Other methods of expansion can also be used, as will be appreciated by those skilled in the art, such as by the use of mechanical means, self-expansion from internal elastic forces, and/or heat-activated forces. Expansion of the balloon 80 can also cause anchors to deploy out from the tension member 30 to engage an interior surface of the ventricular wall 98, as will be described in more detail below. In one embodiment, either transesophageal echocardiography, fluoroscopy or other imaging modalities can be used to position the tension member 30 within the ventricle 92, although any positioning mechanism known in the art can be used. Once the tension member 30 has expanded to its required shape and the tension member 30 has been positioned as needed, the balloon 80 can be deflated and the balloon catheter 82 removed from the ventricle 92 followed by the guidewire 84 and the sheath 86. A person skilled in the art will appreciate that other approaches can be used for delivery of the tension member 30, such as, for example, a transseptal anterograde approach, a coronary sinus anterograde approach, a transvenous approach, and/or a transarterial approach.

In other embodiments, when generally securing a tensioning structure to, for example, the right ventricle or the left ventricle, a guiding catheter or introducing sheath can be used to position the tensioning structure into, for example, the coronary sinus and can be placed through the right atrium or right ventricle during surgical access to the interior of the right atrium. In some embodiments requiring placement of the tension member 30 in the right ventricle, the sheath 86 can be inserted through the superior vena cava. The guidewire 84 can be inserted through the sheath 86 directly into the right atrium and through the tricuspid valve into the right ventricle. The guidewire 84 can be secured at an inferior portion or appendage of the right ventricle by any securing mechanism known in the art. Once the guidewire 84 is in place, the balloon catheter 82 can follow the guidewire 84 through the sheath 86 and into the right ventricle for deployment and placement of the tension member 30 against a right ventricle wall as needed.

Alternatively, a catheter can be percutaneously placed and can be advanced through the right atrial appendage or right ventricle from the inside of the chest cavity. Once a leading end of a tensioning structure is positioned and the corresponding anchoring mechanism secured, the introducing sheath can be retracted, thereby allowing the tensioning structure to expand into the myocardium or against the epicardium of the right atrium or right ventricle. Alternatively, the anchors can be manually set by deforming the anchors using a balloon or other expansion mechanism, as described above. Still further, anchors can be manipulated into contact with the left atrium or left ventricle and secured to provide increased coverage of the tensioning structure around the annulus. Similarly, the guiding catheter or introducing sheath used to position the tensioning structure into the coronary sinus can be used to position the anchors into or through the myocardium of the right atrium or right ventricle. Additional features can be required for any approaches including a puncturing mechanism to penetrate into or through the myocardium, as needed.

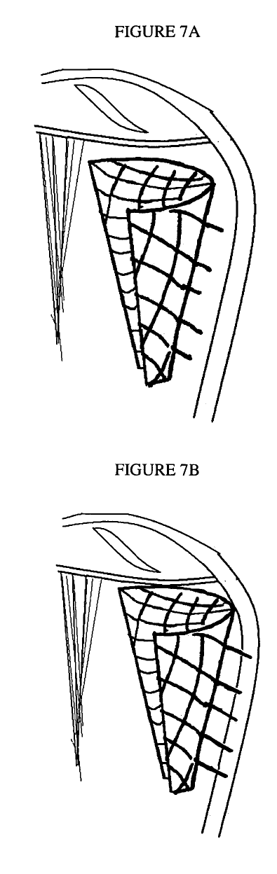

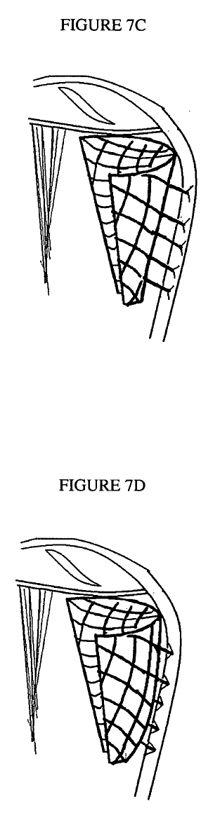

In an embodiment in which the tension member is positioned within the left ventricle 92 of the heart, as described above and as further shown in FIGS. 6A-7C, once the expanded tension member 30 is in position between the papillary muscles 100 below the mitral valve 94, the anchors 34 can presumably be in a tissue piercing configuration in which they extend away from the tension member 30 at an angle greater than about 45 degrees. In this configuration, the anchors 34 can pierce the tissue of the posterior ventricular wall 98, and be inserted therein, as shown in FIG. 7B. Once the anchors 34 are positioned within the tissue, the anchors 34 can take a first securing configuration or open configuration in which attachment features 48a, 48b, associated with the anchors 34, can be deployed. The features 48a, 48b can be deployed such that each feature 48a, 48b extends in a different direction from an elongate portion 50 at an angle that is greater than about 90 degrees and less than about 180 degrees to form an inverted umbrella shape with respect to the elongate portion 50, as shown in FIG. 7C. The anchors 34 can also have a second securing configuration or closed configuration in which the features 48a, 48b can snap or move from the first securing configuration to the second securing configuration such that each feature 48a, 48b extends from the elongate portion 50 at an angle that is less than about 90 degrees and greater than about 0 degrees to form an umbrella shape as shown in FIG. 7D. This snapping or moving from the first securing configuration to the second securing configuration creates a retrograde action on the tissue such that the tissue is pulled or drawn inward and secured against the tension member 30. Once the attachment features 48a, 48b are deployed within the tissue, the tension member 30 also resists pullout. As shown in FIG. 6C, as the attachment features 48a, 48b pull the wall 98 to the tension member 30, the wall 98 can be remodeled to restore the wall's natural and healthy shape.

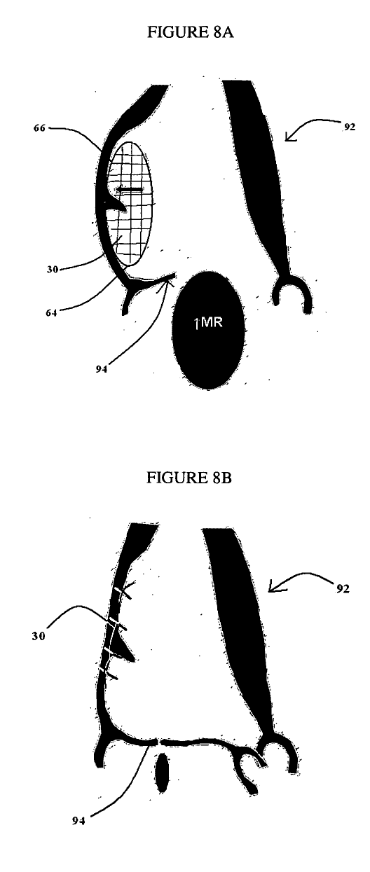

In an embodiment in which the tension member 30 is formed of both the superior tension element 64 and the descending tension element 66, such as the embodiments shown in FIGS. 6A-7C, the superior element 64 can be positioned around a portion of the mitral valve annulus 94 to thereby provide support and facilitate remodeling of the annulus 94. Anchors 34 attached to the superior element 64 can also be deployed to engage tissue around the mitral valve 94. In some embodiments, the superior element 64 can be separate from the descending element 66 and can be attached and secured in a parallel procedure with the descending element 66. As shown in FIGS. 8A and 8B, the use of the tension member 30, having both the superior element 64 and the descending element 66, is effective to remodel the left ventricle 92 and the mitral valve 94 such that the mitral valve leaflets are able to seal properly and the functioning of the mitral valve 94 is returned to a more normal condition. A person skilled in the art will appreciate that various other elements can be included to shape and remodel portions of the heart independently or in combination as needed.

In some embodiments, the tension member 30 and/or the anchors 34 can be formed of or coated with a therapeutic agent. Since the anchors 34 can pierce directly into tissue, therapeutic agents can be beneficially delivered directly into the endocardium, vasculature, myocardium, or epicardium. A therapeutic agent can be directly applied to the tension member 30 and/or the anchors 34 such that it is immediately delivered into the tissue. In general, the tension member 30 and/or the anchors 34 can be coated with or formed of fibrin or other bioabsorbable polymeric matrix capable of delivering therapeutic agents, as known in the art. One or more layers of a therapeutic agent can be applied to the polymeric matrix and can be alternated with layers of the polymeric matrix as needed to accomplish a desired delivery rate and/or delivery amount. More particularly, the adhesion of the coating and the rate at which the drug is delivered can be controlled by the selection of an appropriate bioabsorbable or biostable polymer and by the ratio of drug to polymer in the solution. By this method, therapeutic agents can include but are not limited to drugs such as endothelial growth factors, gene therapies, vasoactive substances, glucocorticoids (e.g. dexamethasone, betamethasone), heparin, hirudin, tocopherol, angiopeptin, aspirin, ACE inhibitors, growth factors, oligonucleotides, statins and, more generally, antiplatelet agents, anticoagulant agents, antimitotic agents, antioxidants, antimetabolite agents, and anti-inflammatory agents. The therapeutic agents can be applied to the tension member 30 and/or the anchors 34, retained on the tension member 30 and the anchors 34 during expansion, and elute the therapeutic at a controlled rate. Other methods of coating and/or forming tension members and anchors with a therapeutic agent to be delivered into the heart will be appreciated by those skilled in the art. Other therapeutic agents and methods related to applying such agents to implantable devices, such as the tension member 30 and the anchors 34 are described in U.S. Application No. 2003/0064965 entitled, “Method of Delivering Drugs to a Tissue Using Drug-Coated Medical Devices,” which is incorporated herein by reference in its entirety. Additional methods and devices for accomplishing therapeutic delivery in a time-release manner are described in U.S. Application No. 2007/0134290 entitled, “Drug Eluting Implantable Medical Device,” also incorporated herein by reference in its entirety.

In other embodiments, the tension member 30 and/or the anchors 34 can be formed of or coated with a material to facilitate introduction of gene therapy directly into the endocardium, vasculature, myocardium, or epicardium. In general, the tension member 30 and/or the anchors 34 can be covered with a polymer composition including fibrin or other composition adapted to provide sustained release of a virus at the chamber wall contacting surface where the tension member 30 is positioned. In one embodiment, the polymer composition can be prepared as a single polymer; as a copolymer, representing two different repeating polymeric units; or as a composition comprising fibrin alone with one or more polymers or one or more other proteins. When the tension member 30 and the anchors 34 are expanded on a balloon, the fibrin is able to expand to accommodate the balloon expansion. The polymer composition can also be a biostable or a bioabsorbable polymer depending on the desired rate of release or the desired degree of polymer stability.

In one embodiment, the tension member 30 and/or the anchors 34 coated with a polymer composition can be loaded with a virus capable of delivering a nucleic acid to a cell within the heart. Preferably, the nucleic acid carried by the virus can have a therapeutic or disease-treating effect on cells that are contacted by the virus delivering the nucleic acid. The nucleic acid delivered by the virus can include a nucleic acid resident within the virus capsid and incorporated during virus assembly in a cell, or the nucleic acid delivered by the virus can be associated on an external portion of the virus. There are a number of viruses, live or inactivate, including recombinant viruses, that can be used to deliver a nucleic acid to the chamber walls and/or the vasculature of the heart, as will be appreciated by those skilled in the art. Those skilled in the art will also recognize that the virus used can be stable enough to infect cells after the virus has been with in contact with the anchors 34 and the tension member 30 and transported in vivo to the delivery sight. Moreover, the virus should be stable at body temperatures for greater than about 24 hours to provide sustained delivery of the virus to the heart.

In one embodiment, the tension member 30 and anchors 34 can be loaded with a virus at the time of formation of the polymer composition by forming the tension member 30 over a balloon and introducing the balloon and tension member 30 into a mold to receive a solution of the polymer composition and the virus. Once the polymer is formed over the tension member 30 and the anchors 34, the tension member 30 and the anchors 34 can be released from the mold. Alternatively or in addition, the virus can be included in a solution as a spray or liquid coating to be applied to the tension member 30 and the anchors 34 at the time of manufactures or by the physician prior to implantation. Other methods associated with coating stents for delivery of gene therapy will be appreciated by those skilled in the art and further details can be found in U.S. Pat. No. 5,833,651 entitled, “Therapeutic Intraluminal Stents,” which is incorporated herein by reference in its entirety. Other examples of gene therapy techniques for delivery into the heart can be found in U.S. Pat. No. 5,792,453 entitled, “Gene Transfer-Mediated Angiogenesis Therapy,” and U.S. Pat. No. 6,508,802 entitled, “Remote Sensing Gene Therapy Delivery Device and Method of Administering a Therapeutic Solution to a Heart,” both of which are incorporated by reference herein in their entireties.

All devices and methods described herein can be configured for permanent placement inside a heart, temporary placement, and resorbable placement as needed. All embodiments can be removable as needed at any time after implantation. The devices and methods described herein can be configured for use in all animals, including human applications, as is preferred, in veterinary applications, and in applications that relate to testing, trials, drug development, etc.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device for stabilizing a ventricle, comprising:
a superior tension member having a substantially arcuate shape and being sized and configured to improve a functioning of atrioventricular valve leaflets;
a descending tension member extending inferiorly from at least a portion of the superior tension member and being shaped to correspond to a wall of a ventricular cavity; and
a plurality of anchors provided at least on the descending tension member, the anchors having attachment features for holding a wall of a ventricular cavity to the descending tension member.

2. The device of claim 1, wherein the superior tension member is a half ring.

3. The device of claim 1, wherein the descending tension member is a mesh formed of intersecting wires.

4. The device of claim 3, wherein the anchors are attached to the mesh at points of intersection between the wires.

5. The device of claim 3, wherein the descending tension member is a mesh formed substantially in the shape of at least a partial cone.

6. The device of claim 5, wherein the anchors are only provided on a portion of the mesh that is configured to contact a wall of a ventricular cavity.

7. The device of claim 5, wherein a portion of the mesh forms the superior tension member.

8. The device of claim 3, wherein each of the plurality of anchors forms an elongate member having a pointed end opposite to an end attached to the mesh, each anchor being positioned in a plane parallel with a plane of the mesh in a delivery configuration.

9. The device of claim 3, wherein each of the plurality of anchors extends from the mesh at an angle greater than about 45 degrees with respect to a plane of the mesh in a piercing configuration.

10. The device of claim 8, wherein the attachment features include at least two attachment elements extending in different directions from the elongate member at an angle greater than about 90 degrees and less than about 180 degrees to grab tissue in a first securing configuration.

11. The device of claim 10, wherein the at least two attachment elements extend in different directions from the elongate member at an angle less than about 90 degrees and greater than about 0 degrees to secure the tissue to the device in a second securing configuration.

12. The device of claim 1, wherein the plurality of anchors have a hooked shape when in a securing configuration within tissue.

13. The device of claim 1, wherein the device is expandable into a deployed shape substantially in the form of a quadrilateral from an introduction shape having a smaller cross-sectional dimension than the deployed shape.

14. The device of claim 1, wherein the device is expandable into a deployed shape substantially in the form of a half cylindrical shape from an introduction shape having a smaller cross-sectional dimension than the deployed shape.

15. The device of claim 1, wherein the device is expandable into a deployed shape substantially in the form of a triangle from an introduction shape having a smaller cross-sectional dimension than the deployed shape.

16. The device of claim 1, wherein the device is formed of nitinol.

17. The device of claim 1, wherein the device is formed of one of deformable stainless steel or spring stainless steel.

18. The device of claim 1, wherein the device is formed of a polymer.

19. The device of claim 1, wherein the device formed of a bioresorbable material.

20. The device of claim 1, wherein the device is coated with a therapeutic agent such that the therapeutic agent can be delivered directly into the myocardium.

21. The device of claim 1, wherein the superior tension member is positioned inferiorly to the mitral valve and the descending tension member is shaped to correspond to a posterior wall of the left ventricle.

22. A method for improving the function of a damaged or diseased heart, comprising:
(a) inserting a device into the ventricle of a patient's heart, wherein the device includes
a superior arcuate member,
a descending element extending inferiorly from the superior arcuate member, and
a plurality of anchors provided at least on the descending element;
(b) locating the superior arcuate member below an atrioventricular valve; and
(c) attaching the anchors to a wall of the ventricle, thereby causing the wall to substantially conform to the descending element.

23. The method of claim 22, wherein the device is inserted in a first, introduction state, and further comprising the step of expanding the device to a second, deployed state within the ventricle.

24. The method of claim 23, wherein the device includes an expandable mesh, the device is inserted over a balloon catheter, and the step of expanding the device includes inflating the balloon.

25. The method of claim 22, wherein the device is inserted into the ventricle using a retrograde transaortic approach.

26. The method of claim 25, wherein the device is inserted over a guidewire anchored in an atrial appendage.

27. The method of claim 22, wherein the device is inserted into the ventricle using a transseptal anterograde approach.

28. The method of claim 22, wherein the device is inserted into the ventricle using a coronary sinus anterograde approach.

29. The method of claim 22, wherein the device is inserted into the ventricle using a transvenous approach.

30. The method of claim 22, wherein the device is inserted into the ventricle using a transarterial approach.

31. The method of claim 22, wherein attaching the anchors includes a step of expanding the anchors from an introduction shape to a deployed shape for piercing tissue.

32. The method of claim 22, wherein attaching the anchors includes a step of piercing a wall of the ventricle and deploying attachment members from the anchors within the wall to form an open configuration.

33. The method of claim 32, wherein attaching the anchors includes a step of moving the attachment members from the open configuration to a closed configuration to pull the tissue against the device.

34. The method of claim 22, further comprising delivering a therapeutic agent directly into the myocardium using a fibrous matrix, wherein the therapeutic agent is one of an endothelial growth factor, a gene therapy, or a vasoactive substance.

35. The method of claim 22, further comprising closing a defect within the ventricular myocardium using a fibrous matrix formed on the device.

36. The method of claim 23, wherein the step of expanding the device includes expanding a device formed of a shape-memory material.

37. The method of claim 22, wherein the superior arcuate member is located below the mitral valve and the anchors are attached to a posterior wall of the left ventricle.

38. A method for remodeling a portion of a patient's heart, comprising:
delivering a tension patch through a vein or artery into a ventricle;
deploying the tension patch within the ventricle; and
anchoring the tension patch on an extended ventricular wall so that the wall is drawn inward.

39. The method of claim 38, wherein delivering the tension patch includes delivering the tension patch using a balloon catheter.
